(19) United States
(12) Patent Application Publication
(10) Pub. No.: US 2023/0148959 A1
(43) Pub. Date: May 18, 2023

(54) Devices and Methods for Sensing Brain Blood Flow Using Head Mounted Display Devices

(71) Applicant: META PLATFORMS TECHNOLOGIES, LLC, Menlo Park, CA (US)

(72) Inventors: Jian XU, Redmond, WA (US); Guohua WEI, Redmond, WA (US)

(21) Appl. No.: 17/528,135

(22) Filed: Nov. 16, 2021

(51) Int. Cl. A61B 5/00; A61B 5/026; G02B 27/01

(52) U.S. Cl. CPC A61B 5/6803; A61B 5/0261; A61B 5/4064; G02B 27/0176; A61B 2562/0238; G02B 2027/0178

(57) ABSTRACT

A head-mounted display device includes a display panel positioned to provide an image toward an eye of a wearer of the head-mounted display device; one or more light sources positioned to provide illumination light toward a head of the wearer; and one or more light detectors for receiving a portion of the illumination light that has been scattered by the head of the wearer so that one or more characteristics associated with a blood flow in the head of the wearer are determined based on the received portion of the illumination light. A method of determining one or more characteristics associated with a blood flow in a head of a wearer of the head-mounted display device is also described.

### TECHNICAL FIELD

This relates generally to head-mounted display devices, and more specifically to head-mounted display devices that are capable of monitoring brain blood flow.

### BACKGROUND

Brain blood flow (also called cerebral blood flow) refers to the movement of blood supplied to a brain. Brain blood flow plays an important role in health and function of the brain. For example, cerebral impairment can be a direct cause of clinical conditions, such as ischemic stroke. However, measuring the brain blood flow typically requires a large medical equipment (e.g., neuroimaging equipment, such as a positron emission tomography scanner or a magnetic resonance imaging scanner), which can prevent real-time on-demand monitoring of the brain blood flow.

### SUMMARY

Thus, there is a need for portable devices that can monitor brain blood flow. Such portable devices may be combined or integrated with head-mounted display devices (also called herein head-mounted displays), which are gaining popularity as means for providing visual information to a user. The devices and methods described herein enable on-demand or daily monitoring of brain blood flow, which may assist with clinical decisions.

In accordance with some embodiments, a head-mounted display device includes a display panel positioned to provide an image toward an eye of a wearer of the head-mounted display device; one or more light sources positioned to provide illumination light toward a head of the wearer; and one or more light detectors for receiving a portion of the illumination light that has been scattered by the head of the wearer. This allows one or more characteristics associated with a blood flow in the head of the wearer to be determined based on the received portion of the illumination light.

In some embodiments, the one or more light detectors include a photodiode.

In some embodiments, the one or more light detectors include a two-dimensional array of photodiodes.

In some embodiments, the one or more light detectors include at least two light detectors that are distinct and separate from each other.

In some embodiments, the one or more light sources include a laser.

In some embodiments, the one or more light sources include an infrared light source.

In some embodiments, the one or more light sources include a visible light source.

In some embodiments, the one or more light sources include a light source providing red light.

In some embodiments, the one or more light sources include a light source providing green light.

In some embodiments, the one or more light sources include at least two light sources that are distinct and separate from each other.

In some embodiments, the one or more light sources and the one or more light detectors are included in a photonic chip.

In some embodiments, the photonic chip includes a photonic waveguide for guiding the illumination light from a light source of the one or more light sources and an optical output coupler for outputting the illumination light from the photonic waveguide.

In some embodiments, the photonic chip includes at least two light sources, at least two light detectors, and at least two photonic waveguides.

In some embodiments, the device includes an eyeglass frame, wherein the photonic chip is located on the eyeglass frame.

In some embodiments, an eyeglass frame with one or more temples, wherein the photonic chip is located on a temple of the one or more temples.

In accordance with some embodiments, a method includes providing, with any head-mounted display device described herein, illumination light toward a head of a wearer of the head-mounted display device for illuminating a region of the head of the wearer; receiving, with the head-mounted display device, a portion of the illumination light that has been scattered by the head of the wearer; and determining one or more characteristics associated with a blood flow in the illuminated region of the head of the wearer based on the received portion of the illumination light.

In accordance with some embodiments, a photonic integrated circuit includes one or more light sources for providing illumination light; one or more light detectors for receiving a portion of the illumination light that has been scattered by a tissue; one or more optical output couplers; and one or more photonic waveguides for guiding the illumination light from the one or more light sources to the one or more optical output couplers.

Thus, the disclosed embodiments provide head-mounted display devices that may monitor brain blood flow.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures. The figures are not drawn to scale unless indicated otherwise.

FIG. 1A is a perspective view of a display device in accordance with some embodiments.

FIG. 1B is a perspective view of an inside of a display device in accordance with some embodiments.

FIG. 2 is a block diagram of a system including a display device in accordance with some embodiments.

FIG. 3 is an isometric view of a display device in accordance with some embodiments.

FIG. 4A is a schematic diagram illustrating a brain blood sensor illuminating human tissue and collecting light back from the human tissue in accordance with some embodiments.

FIG. 4B illustrates an example of light intensity over time in accordance with some embodiments.

FIG. 4C is a schematic diagram illustrating example correlation functions in accordance with some embodiments.

FIG. 4D is an example of a blood flow index over time in accordance with some embodiments.

FIG. 5A is a schematic diagram illustrating a brain blood sensor in accordance with some embodiments.

FIG. 5B is a schematic diagram illustrating a brain blood sensor with one or more waveguides in accordance with some embodiments.

FIG. 5C is a cross-sectional view of the brain blood sensor shown in FIG. 5B in accordance with some embodiments.

FIG. 6 is a flow diagram illustrating a method of collecting light from a head of a wearer for determining one or more characteristics associated a blood flow in the head of the wearer in accordance with some embodiments.

### DETAILED DESCRIPTION

The disclosed embodiments provide wearable devices and methods that enable on-demand or daily monitoring of brain blood flow. The wearable devices may be augmented reality or mixed reality devices. Such augmented reality or mixed reality devices may be worn by users for an extended period of time (e.g., the wearable devices may be all-day wearable devices). In some embodiments, such augmented reality or mixed reality devices may include a varifocal optical assembly. In some implementations, the wearable devices may be virtual reality devices.

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

It will also be understood that, although the terms first, second, etc. are, in some instances, used herein to describe various elements, these elements should not be limited by these terms. These terms are used only to distinguish one element from another. For example, a first light projector could be termed a second light projector, and, similarly, a second light projector could be termed a first light projector, without departing from the scope of the various described embodiments. The first light projector and the second light projector are both light projectors, but they are not the same light projector.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “exemplary” is used herein in the sense of “serving as an example, instance, or illustration” and not in the sense of “representing the best of its kind.”

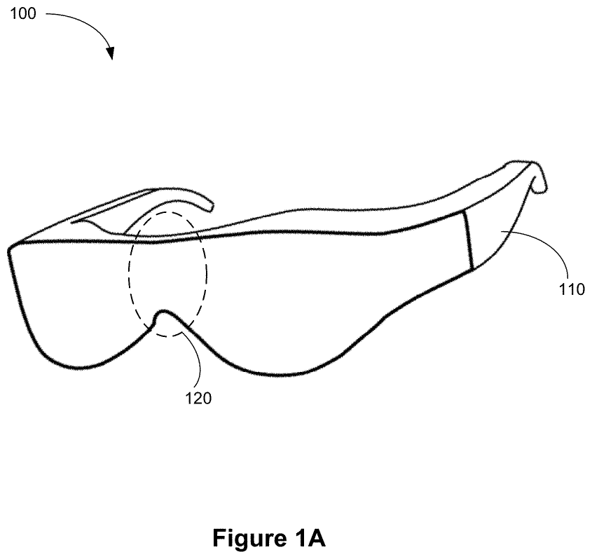

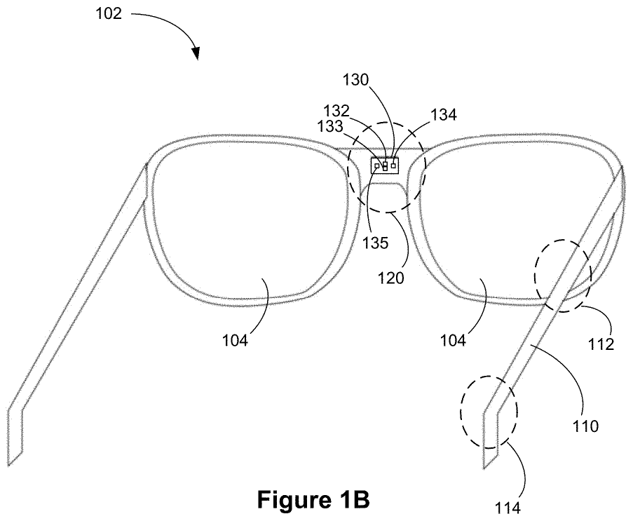

FIG. 1A illustrates display device 100 in accordance with some embodiments. In some embodiments, display device 100 is configured to be worn on a head of a user (e.g., by having the form of spectacles, eyeglasses, or a visor as shown in FIG. 1A) or to be included as part of a helmet that is to be worn by the user. When display device 100 is configured to be worn on a head of a user or to be included as part of a helmet, display device 100 is called a head-mounted display. Alternatively, display device 100 is configured for placement in proximity of an eye or eyes of the user at a fixed location, without being head-mounted (e.g., display device 100 is mounted in a vehicle, such as a car or an airplane, for placement in front of an eye or eyes of the user).

Display device 100 has temples 110 and a nose region 120, on or around which one or more sensors described herein may be located.

FIG. 1B is a perspective view of an inside of another display device 102 in accordance with some embodiments. As shown in FIG. 1B, display device 102 includes one or more displays 104 (also called display panels or electronic displays). Display 104 is configured for presenting visual contents (e.g., augmented reality contents, virtual reality contents, mixed reality contents, or any combination thereof) to a user.

Also as shown in FIG. 1B, in some embodiments, the display device 102 include a blood flow sensor 130 positioned for collecting light from a head of a wearer. In some embodiments, the blood flow sensor 130 is located in the nose region 120 as shown in FIG. 1B. In some embodiments, the blood flow sensor 130 is located on the temple 110 (e.g., location 112 or location 114). In some embodiments, the blood flow sensor 130 is located in the ear region (e.g., on a portion of the temple adjacent to an ear of the wearer when the display device 102 is worn by the wearer). In some embodiments, the display device 102 includes two or more sensors (e.g., a first blood flow sensor 130 in the nose region 120 and a s second sensor on the temple 110; two sensors on the temple; or three or more sensors, etc.).

In some embodiments, the blood flow sensor 130 includes one or more light sources, such as light sources 132 and 133, and one or more light detectors, such as light detectors 134 and 135.

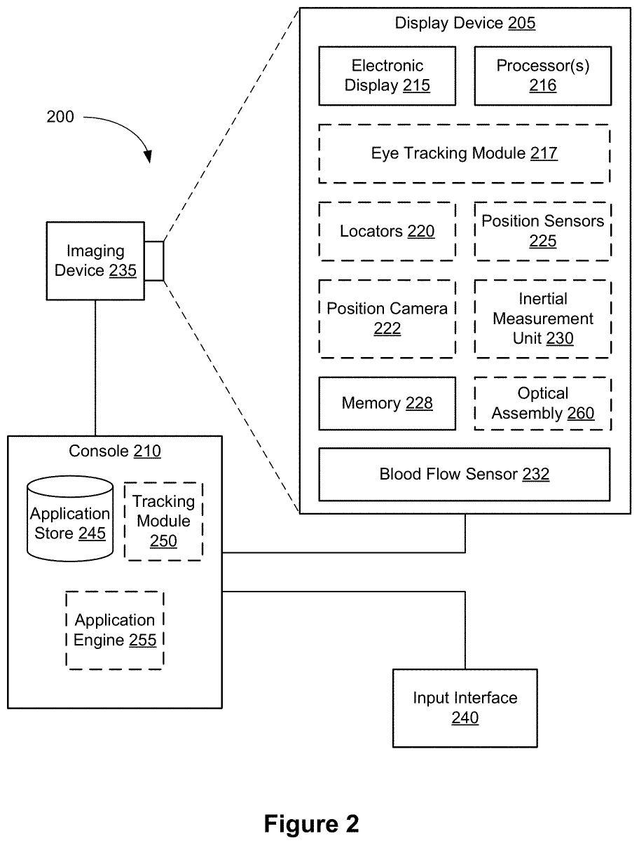

In some embodiments, display device 100 includes one or more components described herein with respect to FIG. 2. In some embodiments, display device 100 includes additional components not shown in FIG. 2.

FIG. 2 is a block diagram of system 200 in accordance with some embodiments. The system 200 shown in FIG. 2 includes display device 205 (which corresponds to display device 100 shown in FIG. 1A or display device 102 shown in FIG. 1B), imaging device 235, and input interface 240 that are each coupled to console 210. While FIG. 2 shows an example of system 200 including one display device 205, imaging device 235, and input interface 240, in other embodiments, any number of these components may be included in system 200. For example, there may be multiple display devices 205 each having associated input interface 240 and being monitored by one or more imaging devices 235, with each display device 205, input interface 240, and imaging devices 235 communicating with console 210. In alternative configurations, different and/or additional components may be included in system 200. For example, in some embodiments, console 210 is connected via a network (e.g., the Internet) to system 200 or is self-contained as part of display device 205 (e.g., physically located inside display device 205). In some embodiments, display device 205 is used to create mixed reality by adding in a view of the real surroundings. Thus, display device 205 and system 200 described herein can deliver augmented reality, virtual reality, and mixed reality.

In some embodiments, as shown in FIGS. 1A and 1B, display device 205 is a head-mounted display that presents media to a user. Examples of media presented by display device 205 include one or more images, video, audio, or some combination thereof. In some embodiments, audio is presented via an external device (e.g., speakers and/or headphones) that receives audio information from display device 205, console 210, or both, and presents audio data based on the audio information. In some embodiments, display device 205 immerses a user in a virtual reality environment.

In some embodiments, display device 205 also acts as an augmented reality (AR) headset. In these embodiments, display device 205 augments views of a physical, real-world environment with computer-generated elements (e.g., images, video, sound, etc.). Moreover, in some embodiments, display device 205 is able to cycle between different types of operation. Thus, display device 205 operate as a virtual reality (VR) device, an augmented reality (AR) device, as glasses or some combination thereof (e.g., glasses with no optical correction, glasses optically corrected for the user, sunglasses, or some combination thereof) based on instructions from application engine 255.

Display device 205 includes electronic display 215 (also called a display panel), one or more processors 216, eye tracking module 217, one or more locators 220, one or more position sensors 225, one or more position cameras 222, memory 228, inertial measurement unit 230, optics 260, and blood flow sensor 232, or a subset or superset thereof (e.g., display device 205 with electronic display 215 and blood flow sensor 232, without any other listed components). Some embodiments of display device 205 have different modules than those described here. Similarly, the functions can be distributed among the modules in a different manner than is described here.

One or more processors 216 (e.g., processing units or cores) execute instructions stored in memory 228. Memory 228 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 228, or alternately the non-volatile memory device(s) within memory 228, includes a non-transitory computer readable storage medium. In some embodiments, memory 228 or the computer readable storage medium of memory 228 stores programs, modules and data structures, and/or instructions for displaying one or more images on electronic display 215.

Electronic display 215 displays images to the user in accordance with data received from console 210 and/or processor(s) 216. In various embodiments, electronic display 215 may comprise a single adjustable display element or multiple adjustable display elements (e.g., a display for each eye of a user).

In some embodiments, the display element includes one or more light emission devices and a corresponding array of spatial light modulators. A spatial light modulator is an array of electro-optic pixels, opto-electronic pixels, some other array of devices that dynamically adjust the amount of light transmitted by each device, or some combination thereof. These pixels are placed behind optics 260 In some embodiments, the spatial light modulator is an array of liquid crystal based pixels in an LCD (a Liquid Crystal Display). Examples of the light emission devices include: an organic light emitting diode, an active-matrix organic light-emitting diode, a light emitting diode, some type of device capable of being placed in a flexible display, or some combination thereof. The light emission devices include devices that are capable of generating visible light (e.g., red, green, blue, etc.) used for image generation. The spatial light modulator is configured to selectively attenuate individual light emission devices, groups of light emission devices, or some combination thereof. Alternatively, when the light emission devices are configured to selectively attenuate individual emission devices and/or groups of light emission devices, the display element includes an array of such light emission devices without a separate emission intensity array.

Optics 260 (also called an optical assembly) direct light from the arrays of light emission devices (optionally through the emission intensity arrays) to locations within each eyebox and ultimately to the back of the user's retina(s). An eyebox is a region that is occupied by an eye of a user located proximity to display device 205 (e.g., a user wearing display device 205) for viewing images from display device 205. In some cases, the eyebox is represented as a 10 mm×10 mm square. In some embodiments, optics 260 include one or more coatings, such as anti-reflective coatings.

In some embodiments, the display element includes an infrared (IR) detector array that detects IR light that is retro-reflected from the retinas of a viewing user, from the surface of the corneas, lenses of the eyes, or some combination thereof. The IR detector array includes an IR sensor or a plurality of IR sensors that each correspond to a different position of a pupil of the viewing user's eye. In alternate embodiments, other eye tracking systems may also be employed.

Eye tracking module 217 determines locations of each pupil of a user's eyes. In some embodiments, eye tracking module 217 instructs electronic display 215 to illuminate the eyebox with IR light (e.g., via IR emission devices in the display element).

A portion of the emitted IR light will pass through the viewing user's pupil and be retro-reflected from the retina toward the IR detector array, which is used for determining the location of the pupil. Alternatively, the reflection off of the surfaces of the eye is used to also determine location of the pupil. The IR detector array scans for retro-reflection and identifies which IR emission devices are active when retro-reflection is detected. Eye tracking module 217 may use a tracking lookup table and the identified IR emission devices to determine the pupil locations for each eye. The tracking lookup table maps received signals on the IR detector array to locations (corresponding to pupil locations) in each eyebox. In some embodiments, the tracking lookup table is generated via a calibration procedure (e.g., user looks at various known reference points in an image and eye tracking module 217 maps the locations of the user's pupil while looking at the reference points to corresponding signals received on the IR tracking array). As mentioned above, in some embodiments, system 200 may use other eye tracking systems than the embedded IR one described herein.

Optional locators 220 are objects located in specific positions on display device 205 relative to one another and relative to a specific reference point on display device 205. A locator 220 may be a light emitting diode (LED), a corner cube reflector, a reflective marker, a type of light source that contrasts with an environment in which display device 205 operates, or some combination thereof. In embodiments where locators 220 are active (i.e., an LED or other type of light emitting device), locators 220 may emit light in the visible band (e.g., about 400 nm to 750 nm), in the infrared band (e.g., about 750 nm to 1 mm), in the ultraviolet band (about 100 nm to 400 nm), some other portion of the electromagnetic spectrum, or some combination thereof.

In some embodiments, locators 220 are located beneath an outer surface of display device 205, which is transparent to the wavelengths of light emitted or reflected by locators 220 or is thin enough to not substantially attenuate the wavelengths of light emitted or reflected by locators 220. Additionally, in some embodiments, the outer surface or other portions of display device 205 are opaque in the visible band of wavelengths of light. Thus, locators 220 may emit light in the IR band under an outer surface that is transparent in the IR band but opaque in the visible band.

Imaging device 235 generates calibration data in accordance with calibration parameters received from console 210. Calibration data includes one or more images showing observed positions of locators 220 that are detectable by imaging device 235. In some embodiments, imaging device 235 includes one or more still cameras, one or more video cameras, any other device capable of capturing images including one or more locators 220, or some combination thereof. Additionally, imaging device 235 may include one or more filters (e.g., used to increase signal to noise ratio). Imaging device 235 is configured to optionally detect light emitted or reflected from locators 220 in a field of view of imaging device 235. In embodiments where locators 220 include passive elements (e.g., a retroreflector), imaging device 235 may include a light source that illuminates some or all of locators 220, which retro-reflect the light towards the light source in imaging device 235. Second calibration data is communicated from imaging device 235 to console 210, and imaging device 235 receives one or more calibration parameters from console 210 to adjust one or more imaging parameters (e.g., focal length, focus, frame rate, ISO, sensor temperature, shutter speed, aperture, etc.).

Input interface 240 is a device that allows a user to send action requests to console 210. An action request is a request to perform a particular action. For example, an action request may be to start or end an application or to perform a particular action within the application. Input interface 240 may include one or more input devices. Example input devices include: a keyboard, a mouse, a game controller, data from brain signals, data from other parts of the human body, or any other suitable device for receiving action requests and communicating the received action requests to console 210. An action request received by input interface 240 is communicated to console 210, which performs an action corresponding to the action request. In some embodiments, input interface 240 may provide haptic feedback to the user in accordance with instructions received from console 210. For example, haptic feedback is provided when an action request is received, or console 210 communicates instructions to input interface 240 causing input interface 240 to generate haptic feedback when console 210 performs an action.

Console 210 provides media to display device 205 for presentation to the user in accordance with information received from one or more of: imaging device 235, display device 205, and input interface 240. In the example shown in FIG. 2, console 210 includes application store 245, tracking module 250, and application engine 255. Some embodiments of console 210 have different modules than those described in conjunction with FIG. 2. Similarly, the functions further described herein may be distributed among components of console 210 in a different manner than is described here.

When application store 245 is included in console 210, application store 245 stores one or more applications for execution by console 210. An application is a group of instructions, that when executed by a processor, is used for generating content for presentation to the user. Content generated by the processor based on an application may be in response to inputs received from the user via movement of display device 205 or input interface 240. Examples of applications include: gaming applications, conferencing applications, video playback application, or other suitable applications.

When tracking module 250 is included in console 210, tracking module 250 calibrates system 200 using one or more calibration parameters and may adjust one or more calibration parameters to reduce error in determination of the position of display device 205. For example, tracking module 250 adjusts the focus of imaging device 235 to obtain a more accurate position for observed locators on display device 205. Additionally, if tracking of display device 205 is lost (e.g., imaging device 235 loses line of sight of at least a threshold number of locators 220), tracking module 250 re-calibrates some or all of system 200.

In some embodiments, tracking module 250 tracks movements of display device 205 using second calibration data from imaging device 235. For example, tracking module 250 determines positions of a reference point of display device 205 using observed locators from the second calibration data and a model of display device 205. In some embodiments, tracking module 250 also determines positions of a reference point of display device 205 using position information from the first calibration data. Additionally, in some embodiments, tracking module 250 may use portions of the first calibration data, the second calibration data, or some combination thereof, to predict a future location of display device 205. Tracking module 250 provides the estimated or predicted future position of display device 205 to application engine 255.

Application engine 255 executes applications within system 200 and receives position information, acceleration information, velocity information, predicted future positions, or some combination thereof of display device 205 from tracking module 250. Based on the received information, application engine 255 determines content to provide to display device 205 for presentation to the user. For example, if the received information indicates that the user has looked to the left, application engine 255 generates content for display device 205 that mirrors the user's movement in an augmented environment. Additionally, application engine 255 performs an action within an application executing on console 210 in response to an action request received from input interface 240 and provides feedback to the user that the action was performed. The provided feedback may be visual or audible feedback via display device 205 or haptic feedback via input interface 240.

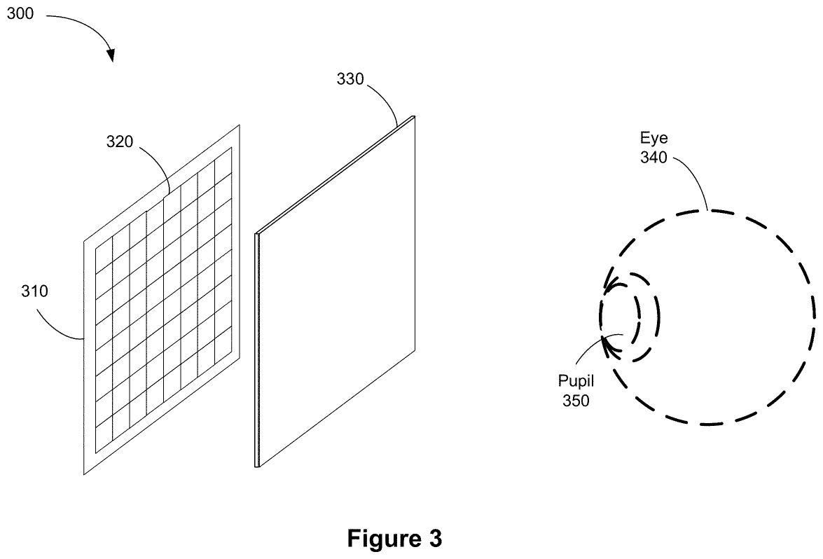

FIG. 3 is an isometric view of display device 300 in accordance with some embodiments. FIG. 3 shows some of the components of display device 205, such as electronic display 215 and optics 260. In some other embodiments, display device 300 is part of some other electronic display (e.g., a digital microscope, a head-mounted display device, etc.). In some embodiments, display device 300 includes light emission device array 310 and optical assembly 330. In some embodiments, display device 300 also includes an IR detector array.

Light emission device array 310 emits image light and optional IR light toward the viewing user. Light emission device array 310 may be, e.g., an array of LEDs, an array of microLEDs, an array of OLEDs, or some combination thereof. Light emission device array 310 includes light emission devices 320 that emit light in the visible light (and optionally includes devices that emit light in the IR).

In some embodiments, display device 300 includes an emission intensity array configured to selectively attenuate light emitted from light emission array 310. In some embodiments, the emission intensity array is composed of a plurality of liquid crystal cells or pixels, groups of light emission devices, or some combination thereof. Each of the liquid crystal cells is, or in some embodiments, groups of liquid crystal cells are, addressable to have specific levels of attenuation. For example, at a given time, some of the liquid crystal cells may be set to no attenuation, while other liquid crystal cells may be set to maximum attenuation. In this manner, the emission intensity array is able to control what portion of the image light emitted from light emission device array 310 is passed to optical assembly 330. In some embodiments, display device 300 uses an emission intensity array to facilitate providing image light to a location of pupil 350 of eye 340 of a user, and minimize the amount of image light provided to other areas in the eyebox.

Optical assembly 330 receives the modified image light (e.g., attenuated light) from emission intensity array (or directly from emission device array 310), and directs the modified image light to a location of pupil 350.

In some embodiments, display device 300 includes one or more broadband sources (e.g., one or more white LEDs) coupled with a plurality of color filters, in addition to, or instead of, light emission device array 310.

Although FIG. 3 shows light emission device array 310 located in front of eye 340, in some implementations, light emission device array 310 may be placed away from eye 340 (e.g., not in front of eye 340). In some implementations, optical assembly 330 includes one or more optical components (e.g., an optical waveguide or a combiner) to relay light from light emission device array 310 (and an emission intensity array, if display device 300 includes the emission intensity array) from light emission device array 310 (that may not be positioned in front of eye 340) toward eye 340.

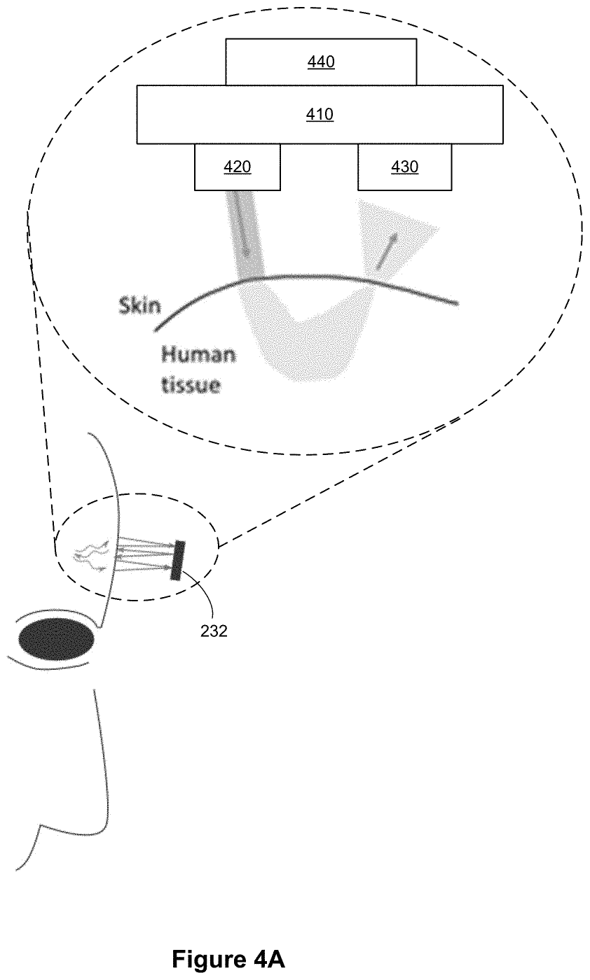

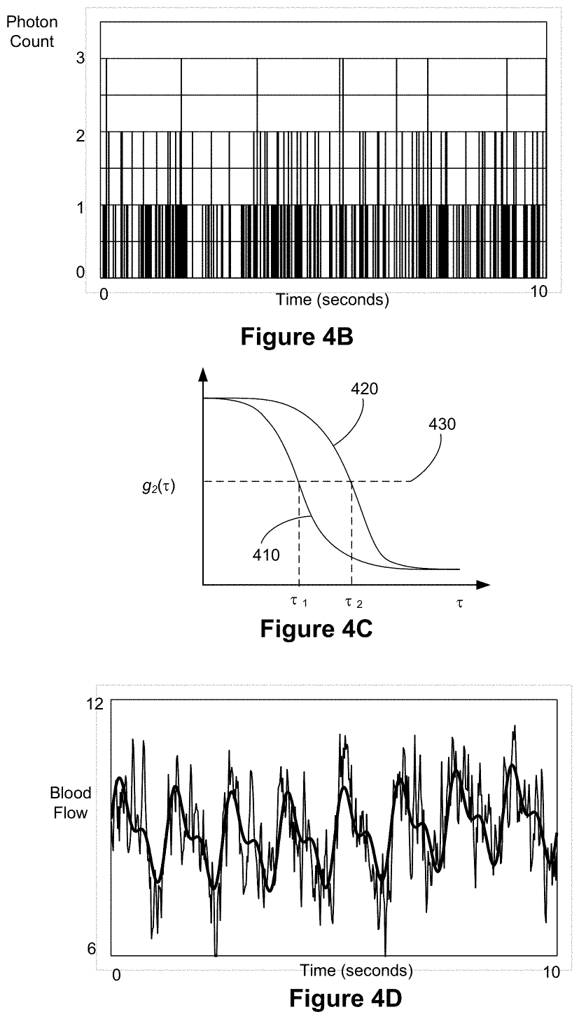

FIG. 4A is a schematic diagram illustrating a brain blood sensor 232 illuminating human tissue (e.g., human skin) and collecting light back from the human tissue in accordance with some embodiments. In some embodiments, the brain blood sensor 232 illuminates human tissue in a head region of a wearer and collects light back from the head region of the wearer, as shown in FIG. 4A.

In some embodiments, the brain blood sensor 232 includes a substrate 410 with one or more light sources 420 and one or more light detectors 430. The one or more light sources 420 emit light onto human tissue (e.g., human skin). In some cases, the emitted light is diffused and scattered by the human tissue, and the one or more light detectors 430 collect light returning from the human tissue (e.g., light that has been back scattered by the human tissue). In some embodiments, the one or more light sources 420 include a coherent light source (e.g., a laser, such as a vertical cavity surface emitting laser or a photonic integrated laser). In some embodiments, an optical output of the coherent light source is continuous. In some embodiments, an optical output of the coherent light source is modulated (e.g., the intensity of the light output from the coherent light source is modulated over time). In some embodiments, the coherent light source has a coherence length of at least 10 m. In some embodiments, the one or more light detectors 430 include a photodiode, a single-photon avalanche photodiode, or a camera (e.g., a multi-pixel camera). In some embodiments, the one or more light detectors 430 are configured for photon counting. The scattered light may form a dynamic speckle pattern which contains information associated with the human tissue. Thus, analyzing time-dependent fluctuation in the intensity of the scattered light can provide time-dependent changes in the human tissue (as in diffuse correlation spectroscopy). In some embodiments, the one or more light detectors 430 include a detector with a large bandwidth to record the intensity fluctuation of one ore more speckle grains in the scattered light. For example, in some implementations, the recorded intensity of the scattered light as a function of time (t) (e.g., the intensity of light as shown in FIG. 4B), I(t), is analyzed to obtain an intensity correlation function, g(τ), which is used to determine a temporal correlation time of I(t). Although FIG. 4B shows the intensity of light over a period of seconds (e.g., ten seconds), a signal intensity of light over any duration (e.g., minutes) may be used.

The cerebral blood flow (CBF) may be determined based on the temporal correlation time of I(t). An example of the temporal correlation operation (or the intensity correlation function) is:

g₂(τ)= [symbol] l(t)l(t−τ)

[symbol]

where [symbol] [symbol] denotes the average over time t. In some embodiments, the temporal correlation is determined over a portion of intensity values of light over time (e.g., tens of milliseconds or hundreds of milliseconds, depending on the user case and the system requirement). FIG. 4C shows example correlation functions: a correlation function 410 for a high blood flow having a short decorrelation time and a correlation function 420 for a low blood flow having a long decorrelation time. The decorrelation time can be determined from the full width half maximum (FWHM) of g₂(τ), or by setting other threshold(s) of g₂(τ) (e.g., the correlation function 410 is characterized by (decorrelation) time τ₁ at which the correlation function 410 crosses a predefined threshold 430, and the correlation function 420 is characterized by (decorrelation) time τ₂ at which the correlation function 420 crosses a predefined threshold 430). As the blood flow rate changes over time, the characteristic (e.g., decorrelation) time of the correlation function changes as the blood flow alters between the high blood flow and the low blood flow. In some implementations, the intensity correlation function g₂(τ) may be converted to field correlation function |g₁(τ)| by using Seigert relation:

$$\vert {g}_{1} ( τ ) \vert = \sqrt{\frac{{g}_{2} ( τ ) - 1}{β}}$$

where β is a constant determined primarily by the collection optics of the experiment, and is equal to one for an ideal experiment setup.

In some embodiments, a blood flow rate (or a blood flow index) is determined based on the decorrelation time or the correlation function. For example, the blood flow index may be determined by fitting g₁(τ) with the following function to determine κ_{D} (τ):

$${g}_{1} ( τ ) = \frac{{r}_{b} exp ( - {κ}_{D} ( τ ) {r}_{1} ) - {r}_{1} exp ( - {κ}_{D} ( τ ) {r}_{b} )}{{r}_{b} exp ( - {κ}_{D} ( 0 ) {r}_{1} ) - {r}_{1} exp ( - {κ}_{D} ( 0 ) {r}_{b} )}$$

where κ_{D}(τ)²=|3μₐ(μₐ+μₛ′)(1+2μₛ′k₀²Fτ/μₐ), r₁²=(lₜᵣ²+ρ²), r₁²=((2z_{b}+lₜᵣ)²+ρ²), lₜᵣ=1/(μₐ+μₛ′), z_{b}=2lₜᵣ (1+R_{eff})/(3(1−R_{eff})), k₀=2πn/λ, μₐ is a tissue absorption coefficient, and μₛ′ is a tissue scattering coefficient. R_{eff} is the effective reflection coefficient (accounting for the refractive index mismatch between the index of refraction of the tissue, n, and the refractive index of a surrounding medium, n₀. ρ is a distance between an illumination source and a detector. Once κ_{D}(τ) is determined, the blood flow index F, which is a measure of blood flow, may be determined from κ_{D}(τ).

FIG. 4D is an example of a blood flow index over time in accordance with some embodiments. The blood flow index (corresponding to the blood flow index F) is determined from g₁(τ) over time. The blood flow index may be expressed in a unit of cm²/s. The blood flow index may be tracked over seconds, minutes, hours, or days, depending on the use case and system requirement. The blood flow index may be determined at a rate of sub-seconds, seconds, minutes, or hours, depending on the use case and system requirement. The blood flow index determined from g₁(τ) (thin line) may contain noises, and thus, in some implementations, filtered blood flow index values (thick line) are used instead.

In some embodiments, the brain blood sensor 232 also includes one or more processors 440 (e.g., microprocessors). In some embodiments, the one or more processors 440 are in communication with the one or more light sources 420 (e.g., the one or more processors 440 are electrically coupled with the one or more light sources 420) for activating the one or more light sources 420 to emit light. In some embodiments, the one or more processors 440 are in communication with the one or more light detectors 430 (e.g., the one or more processors 440 are electrically coupled with the one or more light detectors 430) for receiving signals (e.g., electrical signals) indicating intensity of light received by the one or more light detectors 430. In some embodiments, the one or more processors process the signals for determining one or more characteristics (e.g., heart rate, blood oxygen, cerebral blood flow, etc.) associated with a blood in the head of the wearer. In some embodiments, the one or more processors 440 are in communication with (e.g., in electrical connection with) the processor(s) 216 so that the signals from the one or more light detectors 430 and/or the one or more processors 440) are further processed by the processor(s) 216. In some embodiments, the one or more processors 440 receive and process the signals from the one or more light detectors 430, in which case the brain blood sensor 232 may not include the one or more processors 440.

In some embodiments, information indicating the one or more characteristics associated with the brain blood flow are stored locally only (e.g., only within the display device. In some embodiments, information indicating the one or more characteristics associated with the brain blood flow are communicated with another device (e.g., after encryption and/or anonymization) for comparison of the one or more characteristics associated with the brain blood flow of the wearer with corresponding characteristics associated with brain blood flow for a group of people.

Although the brain blood sensor in FIG. 4A is shown with one light source 420, in some embodiments, the brain blood sensor includes two or more light sources. Similarly, Although the brain blood sensor in FIG. 4A is shown with one light detector 430, in some embodiments, the brain blood sensor includes two or more light detectors.

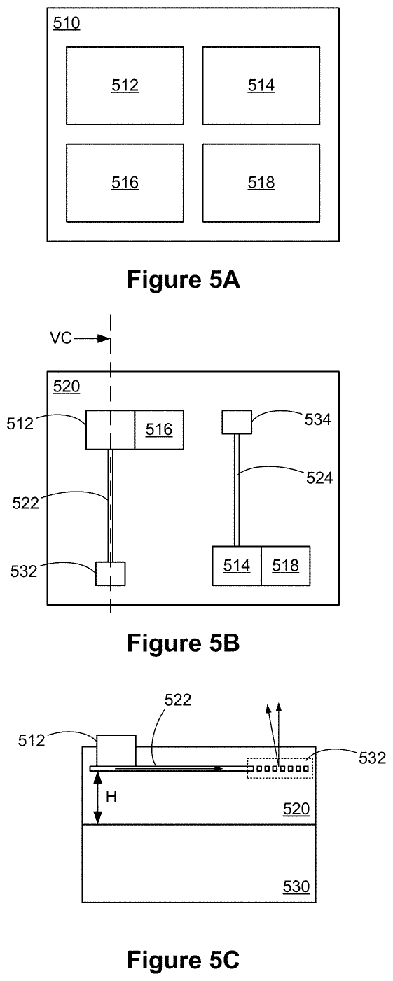

FIG. 5A is a schematic diagram illustrating a brain blood sensor in accordance with some embodiments.

The brain blood sensor shown in FIG. 5A includes light sources 512 and 514 and light detectors 516 and 518. In some embodiments, the light source 512 is configured to emit light having a first wavelength profile and the light detector 516 is configured to detect light having the first wavelength profile. In some embodiments, the light source 514 is configured to emit light having a second wavelength profile and the light detector 518 is configured to detect light having the second wavelength profile. In some embodiments, the first wavelength profile is distinct from the second wavelength profile. In some embodiments, the first wavelength profile is mutually exclusive to the second wavelength profile (e.g., the first wavelength profile does not overlap with the second wavelength profile). In some embodiments, both the first wavelength profile and the second wavelength profile are in an infrared wavelength range (e.g., in a near-infrared wavelength range, such as within 700-1000 nm). In some embodiments, one of the first wavelength profile or the second wavelength profile (e.g., the first wavelength profile) is in an infrared wavelength range (e.g., in a near-infrared wavelength range) and the other of the first wavelength profile or the second wavelength profile (e.g., the second wavelength profile) is not in an infrared wavelength range (e.g., the second wavelength profile is in a visible wavelength range). In some embodiments, the first wavelength profile is within 700-1000 nm and the second wavelength profile is within 600-700 nm. For example, the first wavelength profile includes 940 nm (e.g., from 900-980 nm, 910-970 nm, 920-960 nm, 930-950 nm, etc.) without including 660 nm and the second wavelength profile includes 660 nm (e.g., from 620-700 nm, 630-690 nm, 640-680 nm, 650-670 nm, etc.) without 940 nm. Because oxy-hemoglobin and deoxy-hemoglobin have different absorption coefficients at these wavelengths, a ratio of oxy-hemoglobin and deoxy-hemoglobin may be determined from the intensity of light having the first wavelength profile detected by the light detector 516 and the intensity of light having the second wavelength profile detected by the light detector 518. This, in turn, allows blood oxygen level to be determined from the optical signals (e.g., the intensities of light collected by the light detectors 516 and 518). In some embodiments, the light collected by the light detectors 516 and 518 also includes information indicating a heart rate (also called a heartbeat rate). In some embodiments, the brain blood sensor includes one or more additional light sources (e.g., a light source for a green light) and one or more additional light detectors for determining a heart rate (e.g., by measuring a frequency of fluctuation in absorption of the green light). In some embodiments, the light sources 512 and 514 and the light detectors 516 and 518 are located on a substrate 510.

FIG. 5B is a schematic diagram illustrating a brain blood sensor with one or more waveguides in accordance with some embodiments. In some embodiments, the brain blood sensor is implemented in photonic integrated circuit, as shown in FIG. 5B. The photonic integrated circuit allows integration of multiple optical and electronic components in a small area. For example, a photonic integrated circuit with the light sources and the light detectors may be formed in a 1-mm-by-1-mm area, which can be readily integrated into head-mounted display devices. In some embodiments, the light source 512 is optically coupled with an optical waveguide (also called herein a photonic waveguide) 522 to provide light from the light source 512 to an optical output coupler 532, which emits light delivered via the optical waveguide 522. Similarly, in some embodiments, the light source 514 is optically coupled with an optical waveguide 524 to provide light from the light source 514 to an optical output coupler 534, which emits light delivered via the optical waveguide 524. In some embodiments, the light sources 512 and 514 and the light detectors 516 and 518 are placed on a layer 520.

In some embodiments, at least one of: the optical output coupler 532 or the optical output coupler 534 includes an optical grating, a mirror, or any other diffractive or refractive optical element. In some embodiments, at least one of: an optical grating, a diffractive optical element, or a refractive lens is used for beam shaping (e.g., changing or defining a shape of a beam output from the optical output coupler 532 or 534).

FIG. 5C is a cross-sectional view of the brain blood sensor shown in FIG. 5B in accordance with some embodiments.

In some embodiments, the optical components (e.g., the light sources 512 and 514 and the light detectors 516 and 518, etc.) shown in FIG. 5B are located over a substrate 530. In some embodiments, the substrate 530 is made of silicon (e.g., the substrate 530 includes a portion of a silicon wafer). In some embodiments, the optical waveguide is made of SiN, which may be isolated from the substrate 530 by one or more dielectric layers (e.g., SiO₂ layer having a thickness of H, which may be 2˜3 μm).

In some embodiments, the light source 512, the light source 514, the light detector 516 and light detector 518 are bonded (e.g., flip-chip bonded) or integrated to a photonic integrated circuit in which optical waveguides 522 and 524 and the optical output couplers 532 and 534 are formed.

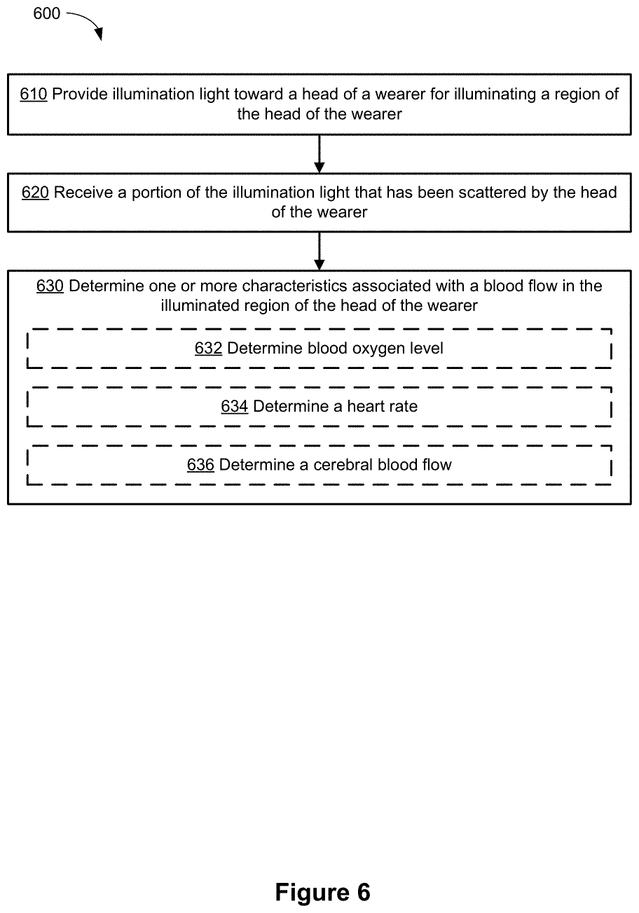

FIG. 6 is a flow diagram illustrating a method 600 of collecting light from a head of a wearer for determining one or more characteristics associated a blood flow in the head of the wearer in accordance with some embodiments.

Method 600 includes (610) providing, with a head-mounted display device described herein, the illumination light toward the head of the wearer for illuminating a region of the head of the wearer (e.g., the light source 420 illuminates a region of the head of the wearer).

Method 600 also includes (620) receiving, with the head-mounted display device, a portion of the illumination light that has been scattered by the head of the wearer (e.g., the light detector 430 receives a portion of the illumination light that has been returned by the human tissue in the head of the wearer).

Method 600 further includes (630) determining one or more characteristics associated with a blood flow in the illuminated region of the head of the wearer based on the received portion of the illumination light (e.g., by processing the optical or electrical signals).

In some embodiments, determining the one or more characteristics associated with the blood flow includes (632) determining blood oxygen level. For example, the blood oxygen level may be determined by comparing the absorption coefficients (or intensities of collected light) at two different wavelengths. In some embodiments, the illumination light includes a first component with a wavelength between 600 nm and 700 nm and a second component with a wavelength between 700 nm and 1000 nm, and determining the one or more characteristics associated with the blood flow includes determining blood oxygen level based on intensities of the first component and the second component in the received portion of the illumination light.

In some embodiments, determining the one or more characteristics associated with the blood flow includes (634) determining a heart rate (also called a heartbeat rate). For example, the heart rate may be determined by determining a frequency of fluctuation in the received light. In some embodiments, the illumination light includes green light, and determining the one or more characteristics associated with the blood flow includes determining a heart rate based on an intensity of the green light in the received portion of the illumination light.

In some embodiments, determining the one or more characteristics associated with the blood flow includes (636) determining a cerebral blood flow. For example, an autocorrelation function of the intensity fluctuation in the received light is determined, which is used, in turn, to determine the cerebral blood flow. In some embodiments, the illumination light includes a near-infrared light, and determining the one or more characteristics associated with the blood flow includes determining a cerebral blood flow based on an intensity of the near-infrared light in the received portion of the illumination light.

In light of these principles, we now turn to certain embodiments.

In accordance with some embodiments, a head-mounted display device (e.g., head-mounted display device 102) includes a display panel (e.g., display 104) positioned to provide an image toward an eye of a wearer of the head-mounted display device; one or more light sources (e.g., light sources 132 and 133) positioned to provide illumination light toward a head of the wearer; and one or more light detectors (e.g., light detectors 134 and 135) for receiving a portion of the illumination light that has been scattered by the head of the wearer.

In some embodiments, the one or more light detectors include a photodiode (e.g., the light detector 516 is a photodiode).

In some embodiments, the one or more light detectors include a two-dimensional array of photodiodes (e.g., the light detector 518 includes a two-dimensional array of photodiodes, such as 100-by-100 array of photodiodes).

In some embodiments, the one or more light detectors include at least two light detectors that are distinct and separate from each other (e.g., light detectors 516 and 518 shown in FIG. 5A).

In some embodiments, the one or more light sources include a laser (e.g., the light source 512 is a laser, such as a VCSEL).

In some embodiments, the one or more light sources include an infrared light source (e.g., the light source 512 is an infrared laser).

In some embodiments, the one or more light sources include a visible light source (e.g., the light source 514 is a visible laser).

In some embodiments, the one or more light sources include a light source providing red light (e.g., for determining blood oxygen level).

In some embodiments, the one or more light sources include a light source providing green light (e.g., for determining a heart rate).

In some embodiments, the one or more light sources include at least two light sources that are distinct and separate from each other (e.g., light sources 512 and 514).

In some embodiments, the one or more light sources and the one or more light detectors are included in a photonic chip (e.g., FIGS. 5B and 5C).

In some embodiments, the photonic chip includes a photonic waveguide (e.g., waveguide 522) for guiding the illumination light from a light source of the one or more light sources and an optical output coupler (e.g., optical output coupler 532) for outputting the illumination light from the photonic waveguide.

In some embodiments, the photonic chip includes at least two light sources, at least two light detectors, and at least two photonic waveguides (e.g., light sources 512 and 514 and light detectors 516 and 518 as shown in FIG. 5B).

In some embodiments, the device includes an eyeglass frame (e.g., the eyeglass frame shown in FIG. 1B). The photonic chip is located on the eyeglass frame.

In some embodiments, an eyeglass frame with one or more temples (e.g., temple 110). The photonic chip is located on a temple of the one or more temples (e.g., location 112, location 114, etc.).

In accordance with some embodiments, a photonic integrated circuit (e.g., the photonic integrated circuit shown in FIGS. 5B and 5C) includes one or more light sources for providing illumination light; one or more light detectors for receiving a portion of the illumination light that has been scattered by a tissue; one or more optical output couplers; and one or more photonic waveguides for guiding the illumination light from the one or more light sources to the one or more optical output couplers.

Although some of various drawings illustrate a number of logical stages in a particular order, stages which are not order dependent may be reordered and other stages may be combined or broken out. While some reordering or other groupings are specifically mentioned, others will be apparent to those of ordinary skill in the art, so the ordering and groupings presented herein are not an exhaustive list of alternatives. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to limit the scope of the claims to the precise forms disclosed. Many modifications and variations are possible in view of the above teachings. The embodiments were chosen in order to best explain the principles underlying the claims and their practical applications, to thereby enable others skilled in the art to best use the embodiments with various modifications as are suited to the particular uses contemplated.

What is claimed is:

1. A head-mounted display device, comprising:
a display panel positioned to provide an image toward an eye of a wearer of the head-mounted display device;
one or more light sources positioned to provide illumination light toward a head of the wearer; and
one or more light detectors for receiving a portion of the illumination light that has been scattered by the head of the wearer so that one or more characteristics associated with a blood flow in the head of the wearer are determined based on the received portion of the illumination light.

2. The device of claim 1, wherein:
the one or more light detectors include a photodiode.

3. The device of claim 1, wherein:
the one or more light detectors include a two-dimensional array of photodiodes.

4. The device of claim 1, wherein:
the one or more light detectors include at least two light detectors that are distinct and separate from each other.

5. The device of claim 1, wherein:
the one or more light sources include a laser.

6. The device of claim 1, wherein:
the one or more light sources include an infrared light source.

7. The device of claim 6, wherein:
the one or more light sources include a visible light source.

8. The device of claim 7, wherein:
the one or more light sources include a light source providing red light.

9. The device of claim 8, wherein:
the one or more light sources include a light source providing green light.

10. The device of claim 1, wherein:
the one or more light sources include at least two light sources that are distinct and separate from each other.

11. The device of claim 1, wherein:
the one or more light sources and the one or more light detectors are included in a photonic chip.

12. The device of claim 11, wherein:
the photonic chip includes a photonic waveguide for guiding the illumination light from a light source of the one or more light sources and an optical output coupler for outputting the illumination light from the photonic waveguide.

13. The device of claim 11, wherein:
the photonic chip includes at least two light sources, at least two light detectors, and at least two photonic waveguides.

14. The device of claim 11, further comprising:
an eyeglass frame, wherein the photonic chip is located on the eyeglass frame.

15. The device of claim 11, further comprising:
an eyeglass frame with one or more temples, wherein the photonic chip is located on a temple of the one or more temples.

16. A method, comprising:
providing, with the head-mounted display device of claim 1, the illumination light toward the head of the wearer for illuminating a region of the head of the wearer;
receiving, with the head-mounted display device, a portion of the illumination light that has been scattered by the head of the wearer; and
determining one or more characteristics associated with a blood flow in the illuminated region of the head of the wearer based on the received portion of the illumination light.

17. The method of claim 16, wherein:
the illumination light includes a first component with a wavelength between 600 nm and 700 nm and a second component with a wavelength between 700 nm and 1000 nm; and
determining the one or more characteristics associated with the blood flow includes determining blood oxygen level based on intensities of the first component and the second component in the received portion of the illumination light.

18. The method of claim 16, wherein:
the illumination light includes green light; and
determining the one or more characteristics associated with the blood flow includes determining a heart rate based on an intensity of the green light in the received portion of the illumination light.

19. The method of claim 16, wherein:
the illumination light includes a near-infrared light; and
determining the one or more characteristics associated with the blood flow includes determining a cerebral blood flow based on an intensity of the near-infrared light in the received portion of the illumination light.

20. A photonic integrated circuit, comprising:
one or more light sources for providing illumination light;
one or more light detectors for receiving a portion of the illumination light that has been scattered by a tissue;
one or more optical output couplers; and
one or more photonic waveguides for guiding the illumination light from the one or more light sources to the one or more optical output couplers.
